(19) United States
(12) Patent Application Publication
(10) Pub. No.: US 2024/0065824 A1
(43) Pub. Date: Feb. 29, 2024

(54) MEDICAL IMPLANT DELIVERY SYSTEM

(71) Applicants: Smith & Nephew, Inc., Memphis, TN (US); Smith & Nephew Orthopaedics AG, Zug (CH); Smith & Nephew Asia Pacific Pte. Limited, SINGAPORE (SG)

(72) Inventor: Allison M. Stauffer, Brighton, MA (US)

(73) Assignees: Smith & Nephew, Inc., Memphis, TN (US); Smith & Nephew Orthopaedics AG, Zug (CH); Smith & Nephew Asia Pacific Pte. Limited, SINGAPORE (SG)

(21) Appl. No.: 18/501,258

(22) Filed: Nov. 3, 2023

Related U.S. Application Data
(63) Continuation of application No. PCT/US2022/027493, filed on May 3, 2022.
(60) Provisional application No. 63/183,949, filed on May 4, 2021.

(51) Int. Cl. A61F 2/08

(52) U.S. Cl. CPC A61F 2/0805

(57) ABSTRACT

Example implant delivery systems are disclosed. An example implant delivery system includes an elongate shaft and a frame coupled to a distal end region of the elongate shaft. The frame includes a body portion and a plurality of attachment arms extending from the body portion, wherein each of the attachment arms includes a support member configured to extend through an aperture of an implant. Further, each of the attachment arms includes a stop disposed on a free end region of the support member, wherein the stop is configured to releasably secure the implant to the frame.

### CROSS REFERENCE TO RELATED APPLICATION

This application is a continuation of International Application No. PCT/US2022/027493, filed May 3, 2022, which claims the benefit of U.S. Patent Application Ser. No. 63/183,949 filed on May 4, 2021, the disclosures of which are incorporated herein by reference.

### TECHNICAL FIELD

The present disclosure pertains generally, but not by way of limitation, to orthopedic implants, implant delivery systems, and methods of treatment. More particularly, the present disclosure relates to a tendon repair implant delivery device for arthroscopic placement of a sheet-like tissue implant over or in the area of a full or partial thickness tear of a tendon, such as the supraspinatus tendon of the shoulder.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. There is an ongoing need to deliver and adequately position medical implants during an arthroscopic procedure in order to treat injuries to the rotator cuff, rotator cuff tendons, or other soft tissue or tendon injuries throughout a body.

### BRIEF SUMMARY

This disclosure provides design, material, manufacturing method, and use alternatives for medical devices.

A first example includes an implant delivery system. The implant delivery system includes an elongate shaft and a frame coupled to a distal end region of the elongate shaft. The frame includes a body portion and a plurality of attachment arms extending from the body portion, wherein each of the attachment arms includes a support member configured to extend through an aperture of an implant. Further, each of the attachment arms includes a stop disposed on a free end region of the support member, wherein the stop is configured to releasably secure the implant to the frame.

Additionally or alternatively, wherein the stop is configured to engage a bottom surface of the implant to releasably secure the frame to the implant.

Additionally or alternatively, wherein the stop is configured to pass through the aperture of the implant.

Additionally or alternatively, wherein the stop is configured to shift from a first configuration prior to passing through the aperture to a second configuration as it passes through the implant, wherein the first configuration is different from the second configuration.

Additionally or alternatively, wherein the stop includes a node extending laterally away from the support member.

Additionally or alternatively, wherein a distal tip portion of the support member extends beyond the node.

Additionally or alternatively, wherein the distal tip portion of the support member is configured to be removed from the node subsequent to attaching the implant to the frame.

Additionally or alternatively, wherein the node includes a first projecting portion extending a laterally away from a first lateral side of the support member and a second projecting portion extending laterally away from a second lateral side of the support member.

Additionally or alternatively, wherein the stop includes an upper face configured to engage a bottom surface of the implant.

Additionally or alternatively, wherein the stop includes a first support arm and a second support arm extending from the first support arm, and wherein the first support arm is positioned at an angle relative to the second support arm.

Additionally or alternatively, wherein the stop is substantially U-shaped.

Additionally or alternatively, wherein the implant is configured to be positioned between the stop and a distal portion of one of the plurality of attachment arms.

Another example implant delivery system includes a tether including a proximal end and a distal end and a frame coupled to the distal end of the tether. Further, the frame includes a body portion and a plurality of attachment arms extending from the body portion, wherein each of the attachment arms includes a support member configured to extend through an aperture of an implant. Additionally, each of the attachment arms includes a stop disposed on a free end region of the support member, wherein the stop is configured to releasably secure the implant to the frame.

Additionally or alternatively, wherein the stop is configured to engage a bottom surface of the implant to releasably secure the frame to the implant.

Additionally or alternatively, wherein retraction of the tether is configured to pull the stop through the aperture of the implant.

Additionally or alternatively, wherein the stop includes a node extending laterally away from the support member.

Additionally or alternatively, wherein a distal tip portion of the support member extends beyond the node.

Additionally or alternatively, wherein the distal tip portion of the support member is configured to be removed from the node subsequent to attaching the implant to the frame.

Additionally or alternatively, wherein the stop includes a first support arm and a second support arm extending from the first support arm, and wherein the first support arm is positioned at an angle relative to the second support arm.

Another implant delivery system includes a frame including an upper surface and a bottom surface and a stabilizer coupled to the frame. Further, the stabilizer is positioned below the bottom surface of the frame and spaced away from the bottom surface of the frame to permit an implant to be releasably secured between the bottom surface of the frame and the stabilizer.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a cross-section of an anterior view of a shoulder of a patient;

FIG. 2 illustrates a shoulder including a head of the humerus mating with the glenoid fossa of the scapula at a glenohumeral joint and an implant being affixed to a tendon using an implant delivery device;

FIG. 3 illustrates an example implant delivery device;

FIG. 4 illustrates the example implant delivery device shown in FIG. 3 attached to an implant;

FIG. 5 illustrates a portion of the implant delivery device shown in FIG. 4;

FIG. 6 illustrates another example implant delivery device;

FIG. 7 illustrates a portion of the implant delivery device shown in FIG. 6 attached to an implant;

FIG. 8 illustrates another example implant delivery device;

FIG. 9 illustrates a portion of the implant delivery device shown in FIG. 8 attached to an implant;

FIG. 10 illustrates another example implant delivery device;

FIG. 11 illustrates a portion of the implant delivery device shown in FIG. 10 attached to an implant;

FIG. 12 is a perspective view of another example implant delivery system;

FIG. 13 is a side view of the implant delivery system shown in FIG. 12.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon and/or promote tissue reformation. Therefore, there is an ongoing need to deliver and adequately position medical implants during an arthroscopic procedure in order to treat injuries to the rotator cuff, rotator cuff tendons, or other soft tissue or tendon injuries throughout a body.

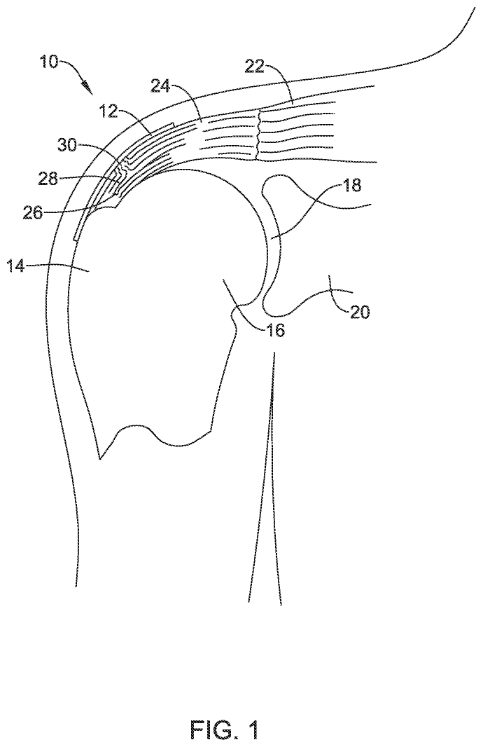

FIG. 1 shows a cross-sectional view of a shoulder 10 including an example implant 12. The shoulder 10 further shows a head 14 of the humerus 16 mating with a glenoid fossa 18 of the scapula 20. The glenoid fossa 18 comprises a shallow depression in the scapula 20. A supraspinatus tendon 22 is also shown. These muscles (along with others) control the movement of the humerus 16 relative to the scapula 20. A distal tendon 24 of the supraspinatus tendon 22 meets the humerus 16 at an insertion point 26.

In FIG. 1, the tendon 24 includes a damaged portion 28 located near the insertion point 26. The damaged portion 28 includes a tear 30 extending partially through the tendon 24. The tear 30 may be referred to as a partial thickness tear. The depicted partial thickness tear 30 is on the bursal side of the tendon, however, the tear may also be on the opposite or articular side of the tendon 24 and/or may include internal tears to the tendon 24 not visible on either surface.

FIG. 1 further illustrates that the tendon repair implant 12 has been placed over the partial thickness tear 30. In this example, the tendon repair implant 12 is placed on the bursal side of the tendon regardless of whether the tear is on the bursal side, articular side or within the tendon. Further, the tendon repair implant 12 may overlay multiple tears.

In some instances, delivery of an implant 12 (e.g., a sheet-like implant) to a target site of a patient may require a physician to create an incision in the patient sufficient to access the target implant site. After creating this access site, the physician may insert an implant delivery system through the access site and position the distal end of the implant delivery system adjacent the target implant site. The physician may then manipulate the implant delivery system to deploy an implant out of a delivery sheath adjacent the target implant site.

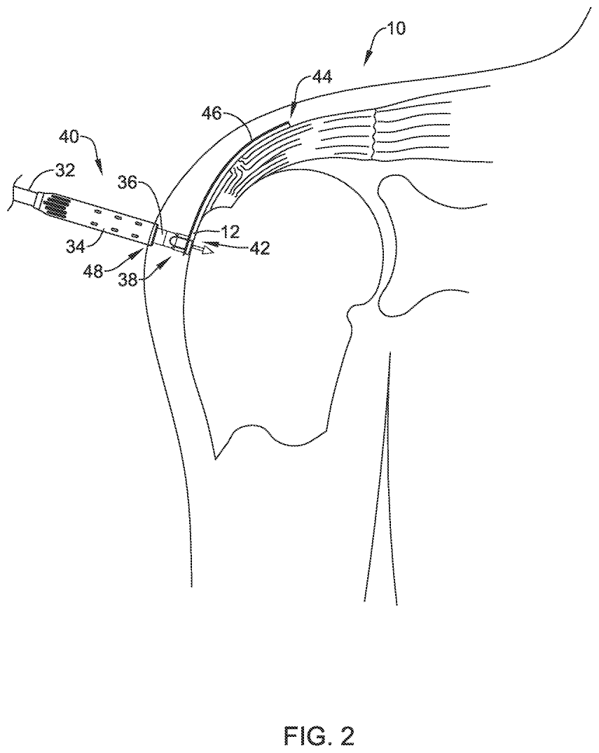

For example, FIG. 2 provides a perspective view of an implant delivery system 40 extending through the shoulder 10 of a patient. FIG. 2 shows the implant delivery system deployed adjacent a target site (e.g., a tear in the supraspinatus tendon). In at least some embodiments, the implant delivery system 40 may include an outer shaft 32 (e.g., a cannula) including a proximal end (not shown), a distal end and a lumen extending within at least a portion of the outer shaft 32. In some examples, the distal end of the outer shaft 32 may be attached to a delivery sheath 34. In other words, the delivery sheath 34 may extend away from the distal end of the outer shaft 32 whereby the distal end of the outer shaft 32 may be attached to a proximal end of the delivery sheath 34. In some examples, the delivery sheath 34 may resemble a substantially cylindrical sheath, a portion of which may be over-molded onto the distal end of the outer shaft 32. As will be described in greater detail below, the delivery sheath 34 may be designed to house the tendon implant 12 in a rolled state as the implant delivery system 40 is advanced to the target site.

FIG. 2 further illustrates that the implant delivery system 40 may include an inner shaft 36 extending within the lumen of the outer shaft 32 and longitudinally movable relative thereto. The inner shaft 36 may include a proximal end (not shown) extending out of the proximal end of the outer shaft 32 and/or otherwise manipulatable relative to the outer shaft 32 by a user. Additionally, in some examples the proximal end of the inner shaft 36 and/or the outer shaft 32 may be coupled to a handle member (not shown). The handle member may be utilized to manipulate the inner shaft 36 relative to the outer shaft 32 and delivery sheath 34. For example, the handle member may be utilized to impart a rotational force to the inner shaft 36 and/or longitudinal movement of the inner shaft 36 relative to the outer shaft 32 and delivery sheath 34.

Additionally, the inner shaft 36 may include a distal end region 38 which is shown extending out of the distal end of the delivery sheath 34 in FIG. 2. Further, the inner shaft 36 may include a lumen extending therein. The lumen of the inner shaft 36 may extend along a portion or the entire length of the inner shaft 36 (e.g., from the distal end region 38 to the proximal end of the inner shaft 36).

The delivery system 40 may further include a frame 46 attached (e.g., detachably attached) to the distal end region 38 of the inner shaft 36. In some instances, the frame 46 may be detachable from the inner shaft 36 in vivo, as described herein. As shown in FIG. 2, the detachable frame 46 may be attached to an implant 12 (e.g., a sheet-like implant) for delivery and deployment at the target site. For purposes of the discussion herein, the combined structure including the frame 46 and the implant 12 may be defined as having a proximal end 42 and a distal end 44 as illustrated in FIG. 2.

When initially positioning the frame 46 and the implant 12 adjacent a target site, a clinician may orient the frame 46 and the implant 12 (for example, via a handle member attached to a proximal portion of the inner shaft 36) such that the proximal portion 42 may be adjacent (e.g., overlaid) on a portion of the humerus (e.g., on the bone), while the distal portion 44 of the frame 46 and the implant 12 may overlay the tendon 24.

As described above, delivery of the implant delivery system 40 may include the insertion of the outer shaft 32 and delivery sheath 34 through an access site (e.g., incision) and advancement to a target site with the detachable frame 46 and the implant 12 contained within a distal portion of the lumen of the delivery sheath 34. After positioning the distal end 48 of the delivery sheath 34 proximate the target site, a clinician may deploy the detachable frame 46 in combination with the implant 12 out of the lumen of the delivery sheath 34, such as by retracting the outer shaft 32 and delivery sheath 34 (via actuation of a handle) relative to the inner shaft 36 and the frame 46, and positioning the implant 12 and the frame 46 over the target site. The frame 46 and the implant 12 may automatically expand to an open state when unconstrained by the delivery sheath 34. As will be illustrated and described in greater detail below, in some examples, the frame 46 may be “shape set” such that its deployed configuration may generally match the curvature of the humeral head. In other words, the frame 46 may expand to a substantially curved configuration which matches the curvature of the humeral head when unconstrained by the delivery sheath 34.

As discussed above, prior to deployment, the detachable frame 46 and the implant 12 combination may be contained (e.g., housed) within the lumen of the delivery sheath 34 for subsequent deployment distally out the distal opening of the delivery sheath 34. The combination of the detachable frame 46 and the implant 12 may wrap and/or fold upon itself such that it may be positioned within the lumen of the delivery sheath 34. Alternatively, the detachable frame 46 and the implant 12 may wrap and/or fold around the implant inner shaft 36 while disposed within the delivery sheath 34.

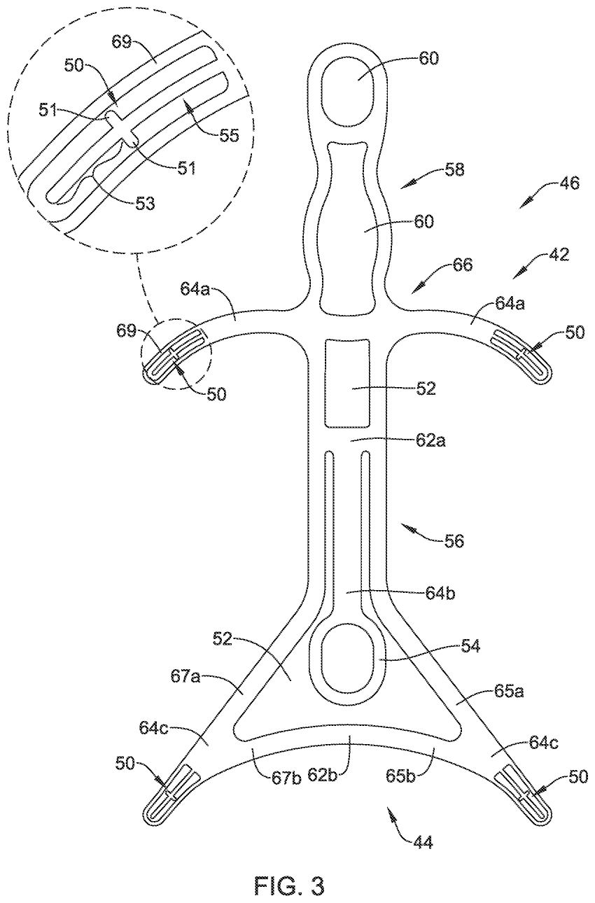

FIG. 3 shows an example detachable frame 46. As shown in FIG. 3, the frame 46 may include a body portion 56. In some examples, the body portion 56 may be understood to define a square, rectangular, circular, ovular, or similarly shaped framework from which other members may extend. For example, the body portion 56 of the frame 46 may bear some resemblance to an elongated rectangle having a proximal portion 42 and a distal portion 44. The body portion 56 may include a first elongated strut spaced apart from a second elongated strut. The body portion 56 may include one or more apertures 52 defined between the struts of the body portion 56. For example, the body portion 56 may include first and second apertures 52 having a stiffening member 62a extending transversely across the body portion between the first and second elongated struts to define the first and second apertures 52. For example, the transverse stiffening member 62a may be located between the first and second apertures 52. The body portion 56 may be positioned along a central portion of an implant 12 when attached thereto. Further, the frame 46 may include a head portion 58 extending away from the proximal portion 42. The head portion 58 may include one or more apertures 60.

As shown in FIG. 3, the detachable frame 46 may include one or more sets of proximal attachment arms 64a and distal attachment arms 64c extending away from body portion 56. It can be appreciated each of the attachment arms 64a/64c, as described herein, may be attached to the implant 12. Further, each respective attachment arm 64a/64c may include a first, proximal end 66 and a second, distal end 68. The first end 66 of each of the attachment arms 64a/64c may be a base end of the attachment arm 64a/64c rigidly attached to the body portion 56, while the opposite, second end 68 may be a free end of the attachment arm 64a/64c spaced away from the body portion 56. In some examples, (such as that shown in FIG. 3), the attachment arms 64a/64c and the head portion 58 may form a monolithic structure with body portion 56. In other words, in some examples the body portion 56, the head portion 58 and the attachment arms 64a/64c may be formed (e.g., machined, cut, shaped, stamped, printed, laser-cut, etc.) as a unitary structure from a single piece of material. In some instances, the detachable frame 46 may be a monolithic structure formed of a superelastic metal material, such as nitinol. However, the above discussion is not intended to be limiting. Rather, it is contemplated that detachable frame 46 may be constructed using alternative materials and/or manufacturing methodologies. For example, the frame 46, or portions thereof, may be constructed from a polymeric material, a ceramic material and/or other various materials. Additionally, the frame 46 may be manufactured via an injection molding or alternative polymer manufacturing methodologies. Alternatively, the frame 46 may be formed through a 3-D printing process, if desired. Further, different portions of the frame 46 (as described above, for example), may be made from a variety of materials and combined using alternative methodologies. For example, the attachment arms 64a/64c may be made from a polymer material and combined with a central frame member constructed from a metal material. Variations of combining different materials with different portions of the frame 46 are contemplated.

FIG. 3 further illustrates that the attachment arms 64a/64c may include a variety of shapes. For example, in some instances, the attachment arms 64a/64c may include a bow and/or general curvilinear shape.

Additionally, FIG. 3 illustrates that the detachable frame 46 may include a connector leg 64b. The connector leg 64b may include a first end attached to the stiffening member 62a, while the opposite, second end of the connector leg 64b may be a free end. Further, as will be described in greater detail below, the connector leg 64b, in combination with the head portion 58, may attach the detachable frame 46 to a portion of the delivery system. Additionally, the connector leg 64b may include one or more features which assist in attaching the frame 46 to the delivery system 40. For example, FIG. 3 illustrates that the connector leg 64b may include an annular portion 54. In some examples, the annular portion 54 may include an aperture positioned in a central region of the annular portion 54. As will be described above, the annular portion 54 of the connector leg 64b may be utilized to attach to a portion of the delivery system 40.

In some examples, the frame 46 may include a variety of shapes and/or geometric arrangements. For example, the frame 46 may include one or more stiffening members 62a/62b extending throughout the frame 46. Further, the stiffening members 62a/62b may be arranged within the frame 46 (e.g., within the body portion 56) such that they create the one or more apertures 52. The number, shape, configuration and/or arrangement of the stiffening members 62a/62b and/or apertures 52 may depend on the particular performance characteristics desired to be imparted to the detachable frame 46. For example, additional stiffening members 62a/62b may be added to frame 46 to provide increased stiffness to frame 46. In other instances, stiffening members 62a/62b may take on particular geometries that increase the stiffness or flexibility in a particular direction while decreasing stiffness or flexibility in a different direction, for example.

The stiffening members 62a/62b may be located (e.g., arranged) throughout the frame 46 in a variety of configurations to provide additional stiffness and/or structural integrity to a particular frame shape. In other words, a wide variety of different shapes and/or arrangements of the stiffening members 62a/62b may be included within the frame 46 to impart customized performance characteristics of the frame 46. For example, in some instances, it may be desirable to transfer rotational forces placed on the head portion 58 to one or more of the attachment arms 64c positioned at the distal portion of the frame 46. The addition of stiffening members 62a/62b may permit transfer of those rotational forces throughout frame 46 (e.g., to the distal portion of frame 46) while minimizing the amount of force lost and/or dissipated throughout the frame 46 due to undesirable flexing of the frame members.

For example, FIG. 3 illustrates that the frame 46 may include a first support strut 65a (e.g., support beam, support member, stiffening strut, etc.) positioned adjacent to a second support strut 65b. Further, the first support strut 65a may converge with the second support strut 65b at the base of the attachment arm 64c. It can be further appreciated that the attachment arm 64c may extend away from the convergence point of the first support strut 65a and the second support strut 65b to a free end of the attachment arm 64c. Similarly, FIG. 3 illustrates that the frame 46 may include a third support strut 67a positioned adjacent to a fourth support strut 67b. Further, the third support strut 67a may converge with the fourth support strut 67b at the base of the attachment arm 64c. It can be further appreciated that the attachment arm 64c may extend away from the convergence point of the first support strut 67a and the second support strut 67b to a free end of the attachment arm 64c. Additionally, it can be appreciated that the second support strut 65b may converge with the fourth support strut 67b to form the stiffening member 62b. It can be appreciated that the combination of the first support strut 65a, the second support strut 65b, the third support strut 67a and the fourth support strut 67b may, collectively, increase the stiffness in the distal portion 44 of the body portion 56 of the frame 46. This increased stiffness may improve the ability of a clinician to control the movement of the frame 46 (when adjacent the target site) from a position outside the body (e.g., as when a clinician is maneuvering the handle from outside a patient's body).

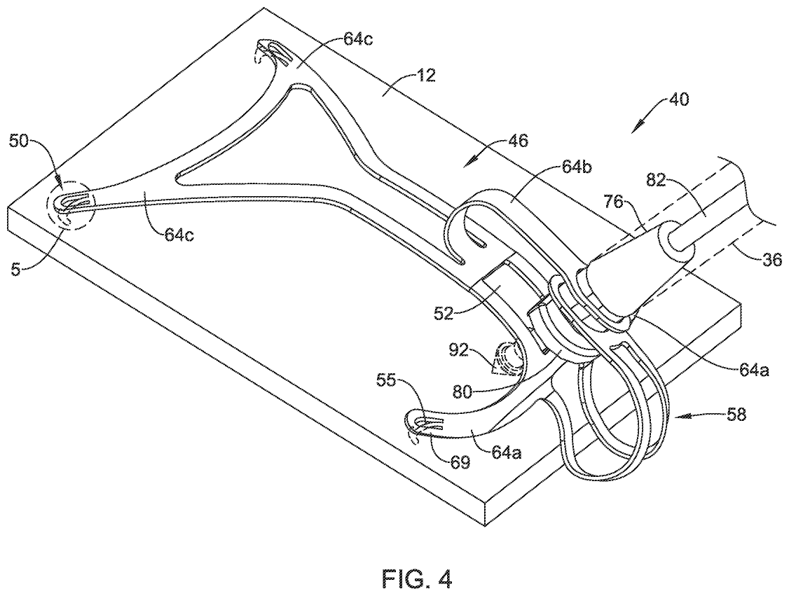

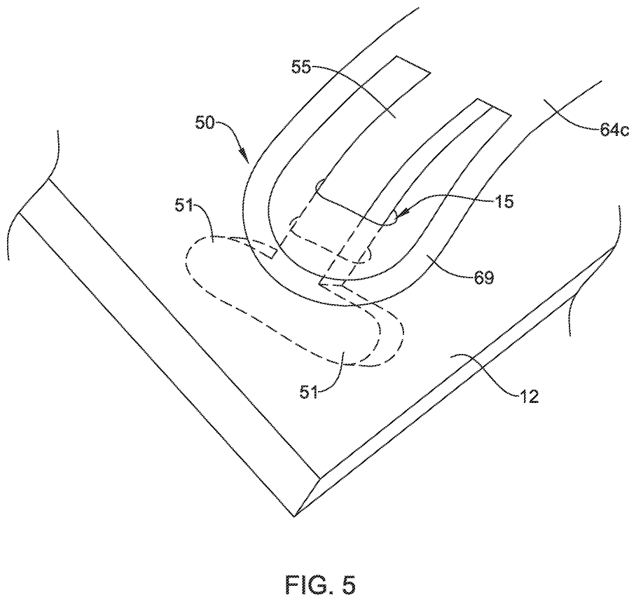

FIG. 3 further illustrates that the frame 46 may include one or more attachment members 50 located along a distal portion of the one or more attachment arms 64a/64c. For example, FIG. 3 illustrates the attachment members 50 positioned at a distal free end of the attachment arms 64a/64c. As will be discussed in greater detail below with respect to FIGS. 4-5, the attachment members 50 may releasably attach the frame 46 to an example implant 12. It can be appreciated that while FIG. 3 shows the attachment members 50 positioned along a distal portion of each of the attachment arms 64a/64c, it is further contemplated that the attachment members 50 may be located along another region of the attachment arms 64a/64c, such as a proximal portion of the attachment arms 64a/64c proximate the body portion 56. In other words, it is contemplated that one or more attachment members 50 may be positioned along any portion of the frame 46.

The detailed view of FIG. 3 further illustrates that each of the attachment members 50 may include a support member 55. The support member 55 may include a proximal portion or end which may be coupled to the frame 46. Further, the detailed view of FIG. 3 illustrates that the support members 55 may include a distal tip portion 53 extending to a free end of the support members 55. Further yet, in some examples, the support members 50 may include one or more nodes 51 (e.g., lobes, projections, bumps, ears, knobs, spurs, etc.) extending laterally away from the support member 55. For example, the nodes 51 may have an enlarged width relative to portions of the support member 55 extending in either direction of the nodes 51. For example, the nodes 51 may have an enlarged width relative to the distal tip portion 53. As disclosed herein, features such as nodes 51 may be designed to releasably secure an implant to a frame and may be referred to as “stops.” As shown in FIG. 3, the support member 55 and associated node 51 may be surrounded by an annular loop portion 69 of the attachment arms 64a/64c.

Additionally, it can be appreciated that the attachment members 50 may be formed as a monolithic structure with the frame 46. In other words, the attachment members 50 may be formed (e.g., laser cut) from the same material as the frame 46. The attachment members 50 and their releasable engagement with the implant 12 are described in greater detail below.

FIG. 4 illustrates the frame 46 coupled to both the implant 12 and various components of the example implant delivery system 40 (e.g., the implant delivery system shown above with respect to FIG. 2). Referring to FIG. 2, the implant delivery system may include an outer shaft 32, whereby a proximal end portion of the outer shaft 32 may be coupled to a handle. Further, as described herein, the distal end of the outer shaft 32 may include a delivery sheath 34. The frame 46 may be positioned within the lumen of the delivery sheath 34 while being advanced to a target site. Further, it can be appreciated that actuation of the handle may retract the outer shaft 32, thereby deploying the frame 46 and implant 12 out of the delivery sheath 34.

As described above, delivery of the implant delivery system 40 may include the insertion of the outer shaft 32 and delivery sheath 34 through an access site (e.g., incision) and advancement to a target site. During advancement to the target site, the detachable frame 46 and the implant 12 (in combination) may be wrapped and/or folded upon itself such that they are positioned within the lumen of the delivery sheath 34. Further, the combination of the detachable frame 46 and the implant 12 may remain wrapped and/or folded within the lumen of the delivery sheath 34 until deployed from the delivery sheath 34.

For example, after positioning the distal end of the delivery sheath 34 proximate the target site, a clinician may deploy the detachable frame 46 (in combination with the implant 12) out of the lumen of the delivery sheath 34, such as by retracting the outer shaft 32 and delivery sheath 34 and positioning the implant 12 and the frame 46 over the target site. Additionally, the frame 46 and the implant 12 may automatically expand to an open state when unconstrained by the delivery sheath 34. Further, in some examples, the frame 46 may be “shape set” such that its deployed configuration may generally match the curvature of the humeral head. In other words, the frame 46 may expand to a substantially curved configuration, with a concave side facing the humeral head which matches the curvature of the humeral head when unconstrained by the delivery sheath 34.

FIG. 4 further illustrates that the implant delivery system 40 described herein may include a tack member 92 designed to secure the delivery system 40 in place prior to a clinician affixing the implant 12 to the bone and/or tendon. For example, FIG. 4 illustrates a tack member 92 extending distally from a tack disk 80. As shown in FIG. 4, the tack member 92 may extend distally from the tack disk 80 and be substantially perpendicular to the implant 12 and/or frame 46.

In some instances, the tack member 92 may resemble a cylindrical pin or rod extending away from the frame 46. Additionally, the tack member 92 may be designed to be rigid enough to be pounded and/or inserted into bone. For example, in some instances, a clinician may apply a force to a proximal portion of the implant delivery system 40 (e.g., on the proximal end of the inner shaft 36) such that the tack member 92 may be “hammered” into a body structure (e.g., bone). As shown in FIG. 4, the tack member 92 may include a tapered distal tip, which may be a sharpened or blunt tapered distal tip in some instances. FIG. 4 illustrates that the tack member 92 may extend through one of the apertures 52 defined in the body portion 56 when the frame 46 is in the deployed configuration of FIG. 4. Furthermore, the tack member 92 may extend through the implant 12 when attached to the frame 46 in the deployed configuration (as shown in FIG. 4).

FIG. 4 further illustrates that the implant delivery system 40 may further include a tether 82 secured to the proximal end of the tack member 92, such as coupled to a tack disk 80, which in turn may be coupled to or formed as a portion of the tack member 92. Therefore, the tack member 92 may be secured to the tether 82 via the tack disk 80. Accordingly, after the tack member 92 has been inserted into bone, retraction of the tether 82 may pull on the tack member 92, thereby releasing it from a target site (e.g., a bone).

Further, the frame 46 may also be coupled to the tether 82 via a connection, such as the combination of the tack disk 80 and a collar 76. It can be appreciated that both the tack disk 280 and the collar 76 may be fixedly attached to the tether 82. Additionally, as discussed above, FIG. 4 illustrates that the tack disk 80 may be coupled to both the head portion 58 and the connector leg 64b of the frame 46. In particular, the head portion 58 and the connector leg 64b may be “sandwiched” between a distal facing surface or rim of the collar 76 and a proximal facing surface or rim of the tack disk 80. In other words, the head portion 58 and the connector leg 64b of the frame 46 may be constrained between the collar 76 and the tack disk 80. Therefore, the frame 46 may be coupled to the tether 82 via being sandwiched between the collar 76 and the tack disk 80.

As described above, the implant delivery system 40 may include an inner shaft 36 which may extend within the lumen of the outer shaft 32 and be longitudinally movable relative thereto. In some examples, the proximal end of the inner shaft 36 and/or the outer shaft 32 may be coupled to the handle. Further, the distal end of the inner shaft 36 may be designed to engage the collar 76. For example, the lumen of the inner shaft 36 may be designed to mate with the outer profile of the collar 76, thereby permitting the distal end of the inner shaft 36 to extend over the collar 76. Accordingly, manipulation of the inner shaft 36 may impart movement to the collar 76 (and consequently, the frame 46). It can be appreciated that the handle may be utilized to manipulate the inner shaft 36 relative to the outer shaft 32 and the delivery sheath 34. For example, the handle may be utilized to impart a rotational force to the inner shaft 36 and/or longitudinal movement of the inner shaft 36 relative to the outer shaft 32 and delivery sheath 34. In some instances, the tether 82 may extend through the lumen of the inner shaft 36 proximally to the handle. The frame 46 may be detachable from the inner shaft 36 such that the inner shaft 36, the outer shaft 32 and handle may be removed while the frame 46 may remain attached to the tether 82.

Additionally, FIG. 4 further illustrates the support member 55 of the one or more attachment members 50 extending through a thickness of the implant 12 to releasably attach the frame 46 to the implant 12. As will be described in greater detail below with respect to FIG. 5, the proximal portion of the support member 55 and the nodes 51 may extend through an aperture of the implant 12 to position the nodes 51 against a bottom surface of the implant 12, whereby the nodes 51 may releasably secure the frame 46 to the implant 12.

FIG. 5 illustrates a detailed view of a portion of implant delivery system 40 of FIG. 4. Specifically, FIG. 5 illustrates a portion of the support member 55 extending through an aperture 15 located in the implant 12 such that the node 51 is positioned below a bottom surface of the implant 12 while the annular loop portion 69 of the support member 55 is positioned above an opposite upper surface of the implant 12. As such, in some instances, the implant 12 may be pressed between the annular loop portion 69 and the node 51. As discussed above, it can be appreciated that the attachment members 50 may be utilized to attach the implant 12 to the frame 46. For example, the distal end portion 53 of each of the attachment members 50 (which extends beyond the node 51) may be passed through the thickness of the implant 12 (e.g., penetrated through the thickness of the implant 12), thus forming apertures 15 through the implant 12. The attachment member 50 may continue to be passed through the thickness of the implant 12 until each of the nodes 51 may be inserted through an aperture 15 of the implant 12 to a bottom surface of the implant 12, whereby each of the nodes 51 of the attachment members 50 may releasably secure the implant 12 to the frame 46. In other words, after being inserted through the aperture 15 of the implant 12, the nodes 51 may engage (e.g., be positioned along) the bottom surface of the implant 12 such that they prevent the implant 12 from being removed from the frame 46 while the implant 12 is being secured to a target site. For example, the nodes 51 may be configured such that they are prevented from being passed back through the aperture 15 until a sufficient retraction force is applied to the frame 46. Accordingly, the implant 12 may remain releasably secured to the frame 46 while the implant 12 is being positioned along the target site. However, after the implant 12 is secured to the target site, a clinician may retract the tether 82, thereby applying a sufficient force to retract the nodes 51 back through the aperture 15 of the implant 12 to the upper surface of the implant 12. Additionally, FIG. illustrates that, in some examples, the proximal portion of the support member 55 may include a curved portion. In some instance, the proximal portion of the support member 55 may be deflected out of the plane of the annular loop portion 69 with the thickness of the implant positioned therebetween.

Additionally, FIG. 5 illustrates that, in some examples, after the distal end portion 53 and the one or more nodes 51 are inserted through the aperture 15 of the implant 12, the distal end portion 53 of the attachment members 50 may be removed. For example, it can be appreciated that the distal end portion 53 of the attachment members 50 may be utilized to penetrate through the implant 12 to form the aperture 15 and thus thread the support member 55 (including the one or more nodes 51) through the aperture 15 of the implant 12, whereby after the nodes 51 are positioned along the underside of the implant 12, the distal end portion 53 of the support member 55 may be removed. For instance, the distal end portion 53 may be cut away from the remainder of the support member 55 and discarded after the implant 12 has been secured to the attachment member 50. In some instances, the implant 12 may be formed with apertures through which the support members may be threaded through.

Further, while FIG. 5 illustrates the attachment member 50 including portions a node 51 extending laterally away from the support member 55 in opposite directions, it can be appreciated that the node 51 may extend from the support member 55 in only one direction. In some instances, the support member 55 may include more than one node 51. For example, the support member 55 may include 2, 3, 4, 5, 6, 7, 8 or more nodes extending away from the support member 55, if desired. Further yet, it can be appreciated that the one or more nodes 51 may include a variety of shapes, including the generally curved/rounded shape illustrated in FIG. 5. For example, each of the nodes 51 may include an ovular, round, square, rectangular, triangular, polygonal, or similar geometric shape. In some examples, the attachment member 50 may include one or more nodes 51 that include dissimilar shapes. For example, the attachment member 50 may include a node 51 that has an ovular shape while also including a node 51 which includes a rectangular shape. Attachment members 50 having nodes 51 which include different shapes and have the same or different relative sizes are contemplated.

As discussed above, the attachment member 50 may be designed to be removably attached to the implant 12. For example, each of the support members 55 may be designed to be pulled (e.g., retracted) through the aperture 15 of the implant 12 after the implant 12 has been positioned and affixed to the target site. It can be appreciated that the aperture 15 may be sized to permit the one or more nodes 51 to be pulled back through the aperture 15 without removing the implant 12 from the target tissue site. For example, once the implant 12 has been affixed to the target site, a user may retract the frame 46 (via pulling on the tether 82), which, in turn, may retract the support members 55 of the attachment members 50 (including the nodes 51) through the aperture 15, thereby releasing the frame 46 from the implant 12. After the frame 46 is released from the implant 12, the implant 12 may remain affixed to the target site while the frame 46 is removed from the body. It can be appreciated that, in some examples, the one or more nodes 51 and/or support members 55 may change shape (e.g., deform) as they are retracted through the aperture 15. For example, the one or more nodes 51 may include a first shape when engaged with the bottom surface of the implant 12 and may shift to a second, different, shape when passing through the aperture 15.

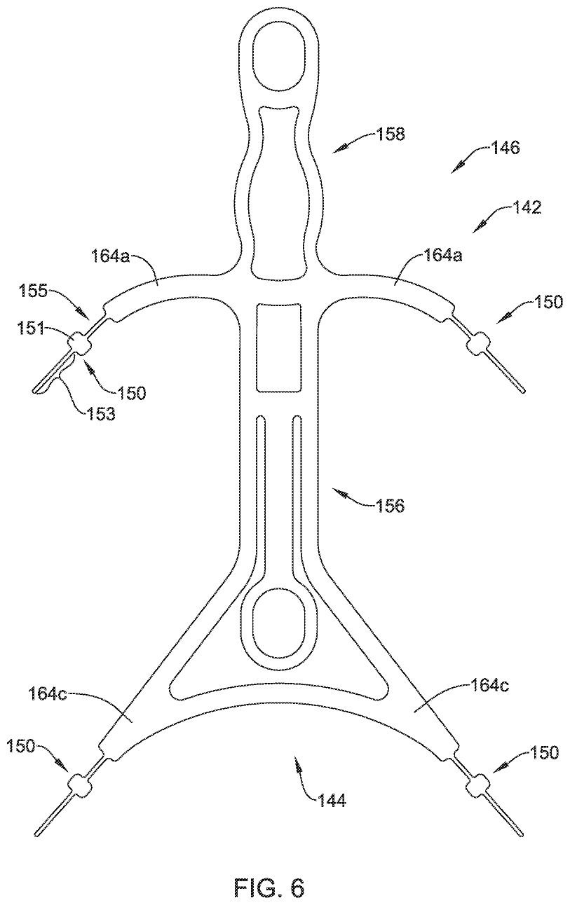

FIG. 6 illustrates another example detachable frame 146. The detachable frame 146 may be similar in form and function to the detachable frame 46 illustrates in FIG. 3. For example, as shown in FIG. 6, the frame 146 may include a body portion 156. In some examples, the body portion 156 may be understood to define a square, rectangular, circular, ovular, or similarly shaped framework from which other members may extend. For example, the body portion 156 of the frame 146 may bear some resemblance to an elongated rectangle having a proximal portion 142 and a distal portion 144. The body portion 156 may include a plurality of struts, stiffening members and apertures like those described for the frame 46 illustrated in FIG. 3. Further, the frame 146 may include a head portion 158 extending away from the proximal portion 142.

It can be appreciated that the frame 146 may be attached to the implant delivery system 40 as described above with respect to FIG. 4. However, as shown in FIG. 6, it can be further appreciated that the shape of the attachment members 150 of the frame 146 may be different from the shape of the attachment members 50 of the frame 46 illustrated in FIG. 3. However, despite having a different shape, the attachment members 150 may function similar to the attachment members 50 described herein. The attachment members 150 and their releasable engagement with the implant 12 are described in greater detail below.

FIG. 6 further illustrates that the frame 146 may include one or more attachment members 150 located along a distal portion of the one or more attachment arms 164a/164c. For example, FIG. 6 illustrates the attachment members 150 positioned at a distal free end of the attachment arms 164a/164c. As discussed herein, the attachment members 150 may be utilized to attach the frame 146 to an example implant 12. It can be appreciated that while FIG. 6 shows the attachment members 150 positioned along a distal portion of each of the attachment arms 164a/164c, it is further contemplated that the attachment members 150 may be located along another region of the attachment arms 164a/164c, such as a proximal portion of the attachment arms 164a/164c proximate the body portion 156. In other words, it is contemplated that one or more attachment members 150 may be positioned along any portion of the frame 146.

FIG. 6 further illustrates that each of the attachment members 150 may include a support member 155. The support member 155 may include a proximal portion or end which is coupled to the frame 146. Further, FIG. 6 illustrates that the support members 150 may include a distal tip portion 153 extending to a free end of the support members 155. Further yet, in some examples, the support members 155 may include a node 151 (e.g., a paddle member) positioned along the support member 155 between a proximal portion of the support member 155 and the distal tip portion 153. The node 151 may extend laterally away from the support member 155. For example, the nodes 151 may have an enlarged width relative to portions of the support member 155 extending in either direction of the nodes 151. For example, the nodes 151 may have an enlarged width relative to the distal tip portion 153. As disclosed herein, features such as nodes 151 may be designed to releasably secure an implant to a frame and may be referred to as “stops.” Additionally, it can be appreciated that the attachment members 150 may be formed as a monolithic structure with the frame 146. In other words, the attachment members 150 may be formed (e.g., laser cut) from the same material as the frame 146.

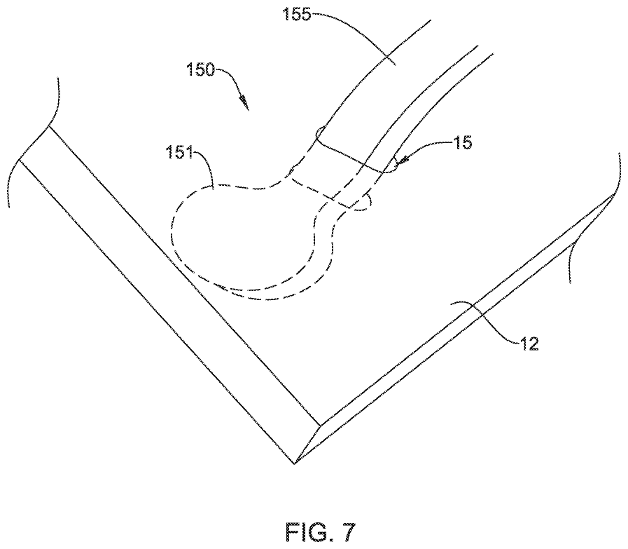

Like FIG. 5 described above, FIG. 7 illustrates the example engagement of the attachment member 150 with the implant 12. Specifically, FIG. 7 illustrates the proximal portion of the support member 155 extending through the thickness of the implant 12, such as extending through an aperture 15 (e.g., an aperture formed by the distal end portion 53 passing through or penetrating the thickness of the implant 12) located in the implant 12 such that the node 151 is positioned below a bottom surface of the implant 12 while the attachment arm 164a/164c of the frame 146 is positioned above an opposite upper surface of the implant 12. As discussed above, it can be appreciated that the attachment members 150 may be utilized to attach the implant 12 to the frame 146. For example, the distal end portion 153 of each of the attachment members 150 (which extends beyond the node 151) may be passed through the thickness of the implant 12 and thus inserted through an aperture of the implant 12 until the nodes 151 are passed through the apertures 15 to a bottom surface of the implant 12, whereby each of the nodes 151 of the attachment members 150 may releasably secure the implant 12 to the frame 146. In other words, after being inserted through the apertures 15 of the implant 12, the nodes 151 may be positioned along the bottom surface of the implant 12 such that they prevent the implant 12 from being removed from the frame 146 while the implant 12 is being secure to a target site. In other words, after being inserted through the aperture 15 of the implant 12, the node 150 may be engage (e.g., be positioned along) the bottom surface of the implant 12 such that they prevent the implant 12 from being removed from the frame 146 while the implant 12 is being secured to a target site. For example, the nodes 151 may be configured such that they are prevented from being passed back through the aperture 15 until a sufficient retraction force is applied to the frame 146. Accordingly, the implant 12 may remain releasably secured to the frame 146 while the implant 12 is being positioned along the target site. However, after the implant 12 is secured to the target site, a clinician may retract the tether 82, thereby applying a sufficient force to retract the nodes 151 back through the aperture 15 of the implant 12 to the upper surface of the implant 12. Additionally, FIG. 5 illustrates that, in some examples, the proximal portion of the support member 155 may include a curved portion.

Additionally, FIG. 7 illustrates that, in some examples, after the distal end portion 153 and the one or more nodes 151 are inserted through the aperture 15 of the implant 12, the distal end portion 153 of the attachment members 150 may be removed. For example, it can be appreciated that the distal end portion 153 of the attachment members 150 may be utilized to penetrate through the implant 12 to form the aperture 15 and thus thread the support member 155 (including the one or more nodes 151) through the aperture 15 of the implant 12, whereby after the nodes 151 are positioned along the bottom surface of the implant 12, the distal end portion 153 of the support member 155 may be removed. For instance, the distal end portion 153 may be cut away from the remainder of the support member 155 and discarded after the implant 12 has been secured to the attachment member 150.

Further, it can be appreciated that the one or more nodes 151 may include a variety of shapes, including the generally rounded shape of the node 151 illustrated in FIG. 7. For example, each of the nodes 151 may include an ovular, round, square, rectangular, triangular, polygonal, spherical, U-shaped, or similar geometric shape. Attachment members 150 having nodes 151 which include different shapes and the same or different relative sizes are contemplated.

As discussed above, the attachment member 150 may be designed to be removably attached to the implant 12. For example, each of the attachment members 150 may be designed to be retracted back through the aperture 15 of the implant 12 after the implant 12 has been positioned and affixed to the target site. It can be appreciated that the aperture may be sized to permit the one or more nodes 151 to be retracted through the aperture without removing the implant 12 from the target tissue site. For example, once the implant 12 has been affixed to the target site, a user may retract the frame 146 (via pulling on the tether 82), which, in turn, may pull the attachment members 150 (including the nodes 151) through the aperture 15, thereby releasing the frame 146 from the implant 12. After the frame 146 is released from the implant 12, the implant 12 may remain affixed to the target site while the frame 146 is removed from the body.

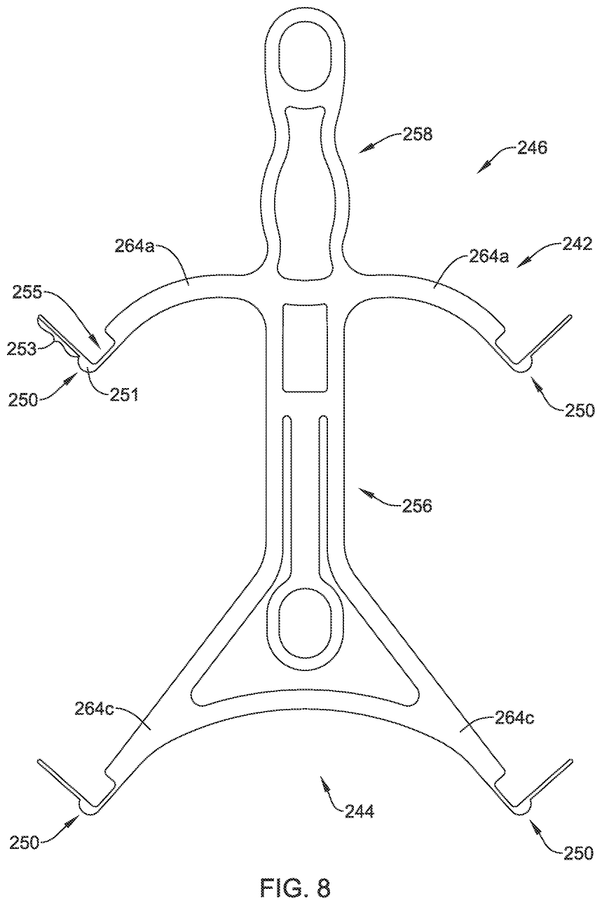

FIG. 8 illustrates another example detachable frame 246. The detachable frame 246 may be similar in form and function to other detachable frames described herein. For example, as shown in FIG. 8, the frame 246 may include a body portion 256. In some examples, the body portion 256 may be understood to define a square, rectangular, circular, ovular, or similarly shaped framework from which other members may extend. For example, the body portion 256 of the frame 246 may bear some resemblance to an elongated rectangle having a proximal portion 242 and a distal portion 244. The body portion 256 may include a plurality of struts, stiffening members and apertures like those described for the frame 46 illustrated in FIG. 3. Further, the frame 246 may include a head portion 258 extending away from the proximal portion 242.

It can be appreciated that that the frame 246 may be attached to the implant delivery system 40 as described above with respect to FIG. 4. However, as shown in FIG. 8, it can be further appreciated that the particular shape of the attachment members 250 of the frame 246 may be different from the shape of other attachment members disclosed herein. However, despite having a different shape, the attachment members 250 may function similar to other attachment members described herein. The attachment members 250 and their releasable engagement with the implant 12 are described in greater detail below.

FIG. 8 illustrates that the frame 246 may include one or more attachment members 250 located along a distal portion of the one or more attachment arms 264a/264c. For example, FIG. 8 illustrates the attachment members 250 positioned at a distal end of the attachment arms 264a/264c. As discussed herein, the attachment members 250 may be utilized to attach the frame 246 to an example implant 12. It can be appreciated that while FIG. 8 shows the attachment members 250 positioned along a distal portion of each of the attachment arms 264a/264c, such as extending from the distal free end of the attachment arms 264/264c, it is further contemplated that the attachment members 250 may be located along another region of the attachment arms 264a/264c, such as a proximal portion of the attachment arms 264a/264c proximate the body portion 256. In other words, it is contemplated that one or more attachment members 250 may be positioned along any portion of the frame 246.

FIG. 8 further illustrates that each of the attachment members 250 may include a support member 255. The support member 255 may include a proximal portion which is coupled to the frame 246. Further, FIG. 8 illustrates that the support members 250 may include a distal tip portion 253 extending to a free end of the support members 255. Further yet, in some examples, the support members 255 may include a node 251 positioned at the distal end region of the support member 255 between a proximal portion of the support member 255 and the distal tip portion 253. The node 251 may extend laterally away from the support member 255. For example, the nodes 151 may have an enlarged width relative to portions of the support member 255 extending in either direction of the nodes 251. For example, the nodes 251 may have an enlarged width relative to the distal tip portion 253. As disclosed herein, features such as nodes 251 may be designed to releasably secure an implant to a frame and may be referred to as “stops.” Additionally, it can be appreciated that the attachment members 250 may be formed as a monolithic structure with the frame 246. In other words, the attachment members 250 may be formed (e.g., laser cut) from the same material as the frame 246.

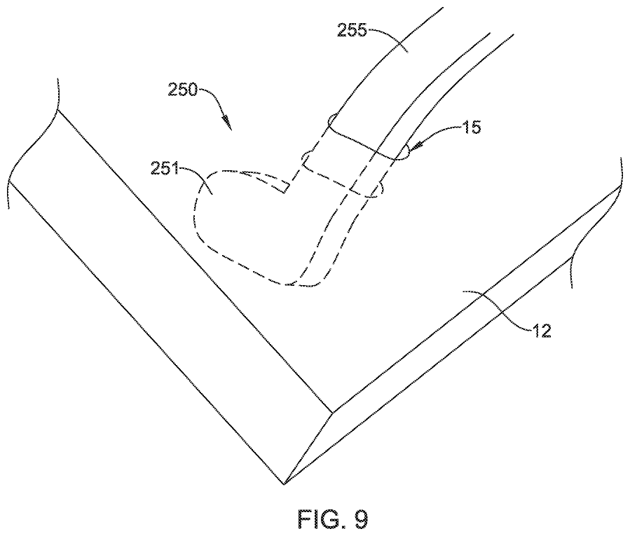

FIG. 9 illustrates the example engagement of the attachment member 250 with the implant 12. Specifically, FIG. 9 illustrates the proximal portion of the support member 255 extending through the thickness of the implant 12, such as extending through an aperture 15 (e.g., an aperture formed by the distal end portion 253 passing through or penetrating the thickness of the implant 12) located in the implant 12 such that the node 251 is positioned below a bottom surface of the implant 12 while the attachment arm 264a/264c of the frame 246 is positioned above an opposite upper surface of the implant 12. As discussed above, it can be appreciated that the attachment members 250 may be utilized to attach the implant 12 to the frame 246. For example, the distal end portion 253 of each of the attachment members 250 (which extends beyond the node 251) may be passed through the thickness of the implant 12 and thus inserted through an aperture 15 of the implant 12 until the nodes 251 are passed through the apertures 15 to a bottom surface of the implant 12, whereby each of the nodes 251 of the attachment members 250 may releasably secure the implant 12 to the frame 246. In other words, after being inserted through the aperture 15 of the implant 12, the nodes 251 may be positioned along the bottom surface of the implant 12 such that they prevent the implant 12 from being removed from the frame 246 while the implant 12 is being secure to a target site. For example, the nodes 251 may be configured such that they are prevented from being passed back through the aperture 15 until a sufficient retraction force is applied to the frame 246. Accordingly, the implant 12 may remain releasably secured to the frame 246 while the implant 12 is being positioned along the target site. However, after the implant 12 is secured to the target site, a clinician may retract the tether 82, thereby applying a sufficient force to retract the nodes 251 back through the aperture 15 of the implant 12 to the upper surface of the implant 12. Additionally, FIG. 5 illustrates that, in some examples, the proximal portion of the support member 255 may include a curved portion.

Additionally, FIG. 9 illustrates that, in some examples, after the distal end portion 253 and the one or more nodes 251 are inserted through the aperture 15 of the implant 12, the distal end portion 253 of the attachment members 250 may be removed. For example, it can be appreciated that the distal end portion 253 of the attachment members 250 may be utilized to penetrate through the implant 12 to form the aperture 15 and thus thread the support member 255 (including the one or more nodes 251) through the aperture 15 of the implant 12, whereby after the node 251 is positioned along the bottom surface of the implant 12, the distal end portion 253 of the support member 255 may be removed. For instance, the distal end portion 253 may be cut away from the remainder of the support member 255 and discarded after the implant 12 has been secured to the attachment member 250.

As shown in FIG. 8, in some instances, the distal end portion 253 may extend at an oblique or perpendicular angle to the proximal portion of the support member 255. Further, it can be appreciated that the one or more nodes 251 may include a variety of shapes, including semi-circular shape of the node 251 illustrated in FIG. 9. For example, each of the nodes 251 may also include ovular, round, square, rectangular, triangular, polygonal, or similar geometric features. Attachment members 250 having nodes 251 which include different shapes and the same or different relative sizes are contemplated.

As discussed above, the attachment member 250 may be designed to be removably attached to the implant 12. For example, each of the attachment members 250 may be designed to be retracted back through the aperture 15 of the implant 12 after the implant 12 has been positioned and affixed to the target site. It can be appreciated that the aperture may be sized to permit the one or more nodes 251 to be retracted through the aperture without removing the implant 12 from the target tissue site. For example, once the implant 12 has been affixed to the target site, a user may retract the frame 246 (via pulling on the tether 82), which, in turn, may pull the attachment members 250 (including the nodes 251) through the aperture 15, thereby releasing the frame 246 from the implant 12. After the frame 246 is released from the implant 12, the implant 12 may remain affixed to the target site while the frame 246 is removed from the body.

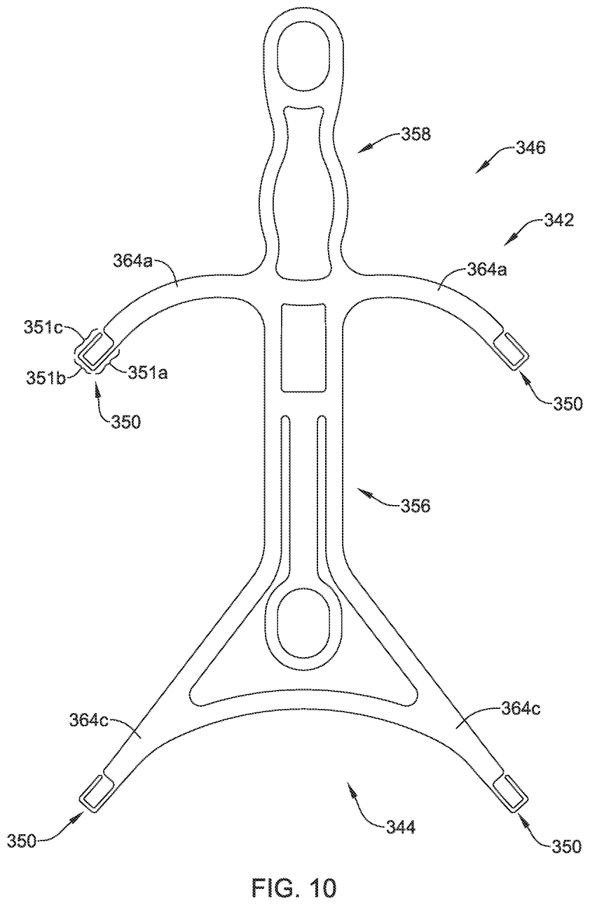

FIG. 10 illustrates another example detachable frame 346. The detachable frame 346 may be similar in form and function to other detachable frames described herein. For example, as shown in FIG. 10, the frame 346 may include a body portion 356. In some examples, the body portion 356 may be understood to define a square, rectangular, circular, ovular, or similarly shaped framework from which other members may extend. For example, the body portion 356 of the frame 346 may bear some resemblance to an elongated rectangle having a proximal portion 342 and a distal portion 344. The body portion 356 may include a plurality of struts, stiffening members and apertures like those described for the frame 46 illustrated in FIG. 3. Further, the frame 346 may include a head portion 358 extending away from the proximal portion 342. However, it can be appreciated that, in some examples, the frame 346 may not include or be devoid of the head portion 358. In other words, the head portion 358 illustrated in FIG. 10 may be optional in some embodiments.

It can be appreciated that that the frame 346 may be attached to the implant delivery system 40 as described above with respect to FIG. 4. However, as shown in FIG. 10, it can be further appreciated that the shape of the attachment members 350 of the frame 346 may be different from the shape of other attachment members disclosed herein. However, despite having a different shape, the attachment members 350 may function similar to other attachment members described herein. The attachment members 350 and their releasable engagement with the implant 12 are described in greater detail below.

FIG. 10 illustrates that the frame 346 may include one or more attachment members 350 located along a distal portion of the one or more attachment arms 364a/364c. For example, FIG. 10 illustrates the attachment members 350 positioned at a distal free end of the attachment arms 364a/364c. The attachment members 350 may have a width substantially less than the width of the attachment arms 364a/364c from when the attachment members 350 extend from. As discussed herein, the attachment members 350 may be utilized to attach the frame 346 to an example implant 12. It can be appreciated that while FIG. 10 shows the attachment members 350 positioned along a distal portion of each of the attachment arms 364a/364c, it is further contemplated that the attachment members 350 may be located along another region of the attachment arms 364a/364c, such as a proximal portion of the attachment arms 364a/364c proximate the body portion 356. In other words, it is contemplated that one or more attachment members 350 may be positioned along any portion of the frame 346.

FIG. 10 further illustrates that each of the attachment members 350 may include a first support member 351a, a second support member 353b and a third support member 351c. As illustrated in FIG. 10, the first support member 351a may include a proximal portion or end which is coupled to the frame 346. The second support member 351b may extend between the first support member 351a and the third support member 351c. The third support member 351c may extend from the second support member 351b to a free end of the attachment member 350. Additionally, it can be appreciated that the attachment members 350 may be formed as a monolithic structure with the frame 346. In other words, the attachment members 350 may be formed (e.g., laser cut) from the same material as the frame 346.

FIG. 10 further illustrates that the combination of the first support member 351a, the second support member 351b and the third support member 351c may be generally configured to form a “U” or a “hook” shape. In some examples, it can be appreciated that the second support member 351b may extend from the first support member 351a at an oblique or perpendicular angle. Additionally, it can be further appreciated that the third support member 351c may extend from the second support member 351b at an oblique or perpendicular angle. Further, it is contemplated that the combination of the first support member 351a, the second support member 351b and the third support member 351c may be formed into other shapes.

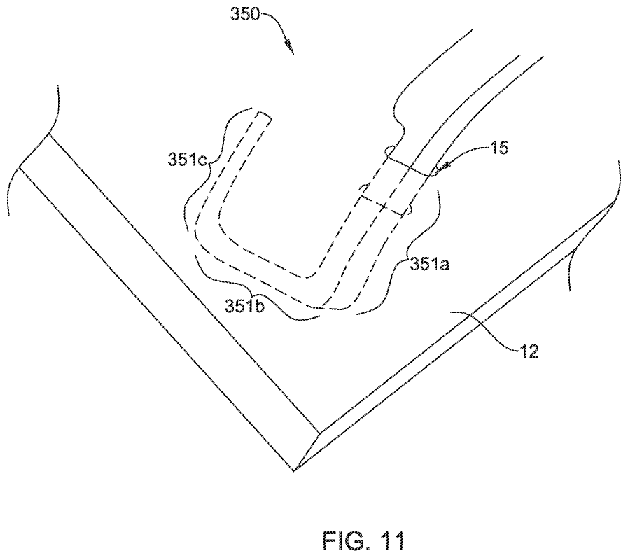

FIG. 11 illustrates the example engagement of the attachment member 350 with the implant 12. Specifically, FIG. 11 illustrates the attachment member 350 (including the combination of the first support member 351a, the second support member 351b and the third support member 351c) may be passed through or penetrated through the thickness of the implant 12 (thereby forming an aperture) and thus inserted through an aperture 15 of the implant 12 with the first support member 351a extending through the aperture 15 located in the implant 12. In some instance, the attachment member 350 may be deflected from its equilibrium, unbiased shape, shown in FIG. 10, to a deflected, straightened shape for penetrating through the thickness of the implant 12. For instance, the second support member 351b may be deflected relative to the first support member 351a to increase the angle between the first support member 351a and the second support member 351b and/or the third support member 351c may be deflected relative to the second support member 351b to increase the angle between the second support member 351b and the third support member 351c, to thereby straighten the attachment members 350 to facilitate inserting the attachment member 350 through the aperture 15 of the implant 12. After being inserted through the thickness of the implant 12 (e.g., through the aperture 15 of the implant 12), the attachment member 350 may revert back to or toward its equilibrium, unbiased shape bent shape (e.g., “U” or “hook” shape). For instance, the angle between the first support member 351a and the second support member 351b may be reduced to or toward its oblique or perpendicular angle in the equilibrium state and/or the angle between the second support member 351b and the third support member 351c may be reduced to or toward its oblique or perpendicular angle in the equilibrium state. It can be appreciated that the bent shape of the first support member 351a, the second support member 351b and the third support member 351c may be designed to releasably secure an implant to a frame and may be referred to as “stops.”

Additionally, it can be appreciated from FIG. 11 that after being inserted through the aperture 15 of the implant 12, the first support member 351a (or a portion thereof) may remain positioned within the aperture 15, while the second support member 351b and the third support member 351c may be positioned along the bottom surface of the implant 12 while the remainder of the frame 346 is positioned along the upper surface of the implant such that the shaped arrangement of the combination of the first support member 351a, the second support member 351b and the third support member 351c prevent the implant 12 from being removed from the frame 346 until after the implant 12 is secured to a target site.

As discussed above, the attachment member 350 may be designed to be removably attached to the implant 12. For example, each of the attachment members 350 may be designed to be retracted back through the aperture 15 of the implant 12 after the implant 12 has been positioned and affixed to the target site. It can be appreciated that the aperture may be sized to permit the combination of the first support member 351a, the second support member 351b and the third support member 351c to be retracted back through the aperture 15 without removing the implant 12 from the target tissue site. For example, once the implant 12 has been affixed to the target site, a user may retract the frame 346 (via pulling on the tether 82), which, in turn, may pull the attachment members 350 (including the combination of the first support member 351a, the second support member 351b and the third support member 351c) through the aperture 15, thereby removing the frame 346 from the implant 12. In some instances, the attachment members 350 may be straightened as they are being pulled back through the apertures 15. For example, the second support member 351b and the third support member 351c may be deflected relative to the first support member 351a to increase the angle between the first support member 351a and the second support member 351b (and to also increase the angle between the second support member 351b and the third support member 351c), to thereby straighten the attachment members 350. After the frame 346 is removed from the implant 12, the implant 12 may remain affixed to the target site while the frame 346 is removed from the body.

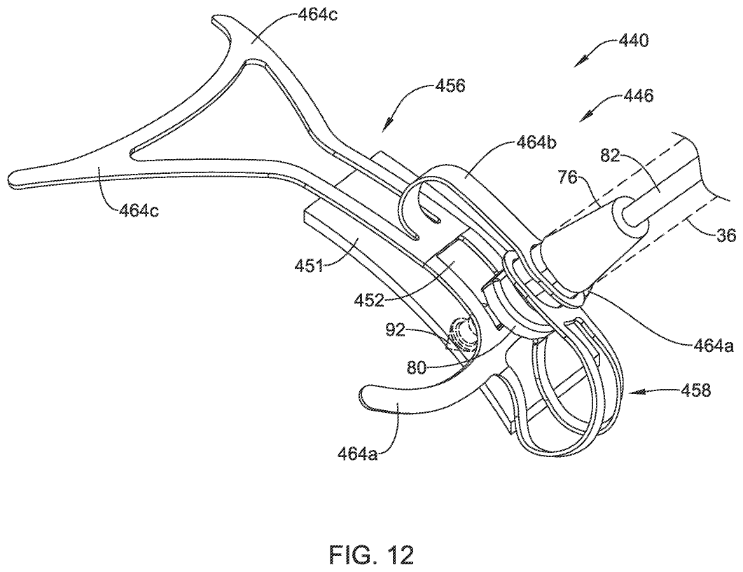

FIG. 12 is a perspective view of another implant delivery system 440. The implant delivery system 440 may include an example detachable frame 446. The detachable frame 446 may be similar in form and function to the detachable frame 46 illustrates in FIG. 3. For example, as shown in FIG. 12, the frame 446 may include a body portion 456 including a plurality of struts, stiffening members and apertures like those described for the frame 46 illustrated in FIG. 3. Additionally, the detachable frame 446 may include one or more sets of proximal attachment arms 464a and distal attachment arms 464c extending away from body portion 456.

Further, as described herein with respect to FIG. 4, the frame 446 may include a head portion 458 and a connector leg 464b, each of which may include features (e.g. apertures) which may be utilized to couple the frame 446 to one or more components of the implant delivery system 440. For example, as described herein, the head portion 458 and the connector leg 464b may be “sandwiched” between a distal facing surface or rim of the collar 46 and a proximal facing surface or rim of the tack disk 80. In other words, the head portion 458 and the connector leg 464b of the frame 446 may be constrained between the collar 76 and the tack disk 80.

Additionally, FIG. 12 illustrates that both the tack disk 80 and the collar 76 may be fixedly attached to a tether 82 (as described herein with respect to FIG. 4). Therefore, the frame 446 may also be coupled to a tether 82 via the combination of the tack disk 80 and the collar 76 (e.g., the frame 446 may be coupled to a tether 82 via being sandwiched between the collar 76 and the tack disk 80).

Further, like that described with respect to FIG. 4, FIG. 12 illustrates that the implant delivery system 440 described herein may include a tack member 92 designed to secure the delivery system 440 in place prior to a clinician affixing the implant 12 to the bone and/or tendon. For example, FIG. 12 illustrates a tack member 92 extending distally from a tack disk 80. As shown in FIG. 12, the tack member 92 may extend distally from the tack disk 80 and be substantially perpendicular to the implant 12 and/or frame 446.

FIG. 12 further illustrates that the implant delivery system 440 may include a stabilizer 451 positioned below (e.g., underneath) the bottom surface of the frame 446. The stabilizer 451 may extend along a portion the length of the frame 446. For example, the stabilizer 451 may include a first end positioned adjacent to the head portion 458 and a second end positioned along the body portion 456. However, it is contemplated that the stabilizer 451 may include a variety of lengths along the frame 446. For example, the stabilizer 451 may include a first end positioned adjacent to the head portion 458 and a second end which is positioned adjacent to the distal end region of the frame 446. It can be appreciated that the stabilizer 451 may include a first end positioned adjacent to the head portion 458 and a second end which is positioned adjacent to any portion the frame 446 distal to the tack member 92.

Additionally, FIG. 12 illustrates that the stabilizer 451 may be positioned between the frame 446 and an upper rim of the tack member 92. It can be appreciated that the dotted outline of the tack member 92 in FIG. 12 depicts that portion of the tack member 92 as positioned underneath the stabilizer 451. In other words, the implant delivery system 440 may be designed such that the stabilizer 451 is contained between the portion of the tack member 92 and the underside of the frame 446. In some instances, the stabilizer 451 is directly attached to the tack member 92. In other instances, the stabilizer is directly attached to the frame 446, such as formed as a monolithic portion of the frame 446.

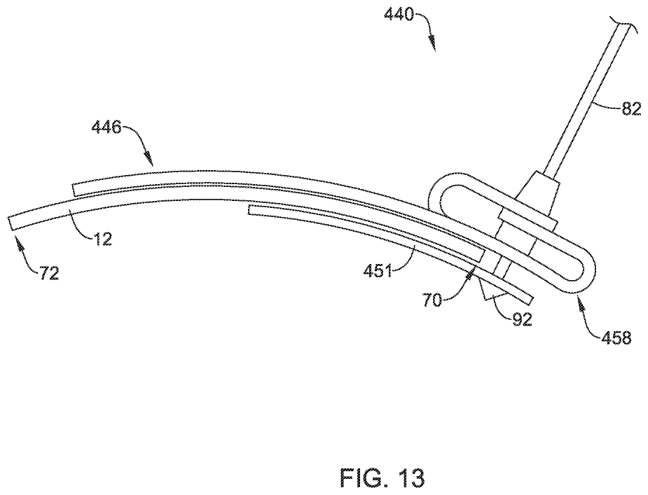

FIG. 13 is a side view of the stent delivery system 440 described with respect to FIG. 12. As described above, FIG. 13 illustrates the stabilizer 451 positioned between the upper rim of the tack member 92 and the bottom surface of the frame 446. Additionally, FIG. 13 illustrates that, in some examples, the implant 12 may be positioned between an upper surface of the stabilizer 451 and the bottom surface of the frame 446. For example, the stabilizer 451 may be spaced away from the bottom surface of the frame 446 such that it defines a gap (e.g., space, opening, etc.) to releasably secure the implant 12. In other words, in some examples, the implant 12 may be sandwiched between the upper surface of the stabilizer 451 and the bottom surface of the frame 446.

Additionally, FIG. 13 illustrates that the implant may include a first end 70 which is positioned distal to the tack member 92. Therefore, it can be appreciated that the tack member 92 may not extend through a portion of the implant 12, rather the tack member 92 may extend along a distal edge of the implant 12. Accordingly, the frame 446 may be releasably secured to the implant 12. For example, after the implant 12 has been affixed to a target site (e.g., bone) a physician may retract the tether 82, thereby sliding the frame 446 away from (e.g., off the proximal end of) the implant 12. It can be appreciated that, in this example, the proximal attachment arms 464a and distal attachment arms 464c may not be fixedly attached to the implant 12. Rather, the proximal attachment arms 464a and distal attachment arms 464c may lay atop the upper surface of the implant 12, potentially providing a downward force which, in combination with the stabilizer 451, releasably secures the implant 12 between the bottom surface of the frame 446 and the upper surface of the stabilizer 451.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An implant delivery system, the implant delivery system comprising:
an elongate shaft; and
a frame coupled to a distal end region of the elongate shaft, the frame including a body portion and a plurality of attachment arms extending from the body portion, wherein each of the attachment arms includes a support member configured to extend through an aperture of an implant;
and wherein each of the attachment arms includes a stop disposed on a free end region of the support member, wherein the stop is configured to releasably secure the implant to the frame.

2. The implant delivery system of claim 1, wherein the stop is configured to engage a bottom surface of the implant to releasably secure the frame to the implant.

3. The implant delivery system of claim 1, wherein the stop is configured to pass through the aperture of the implant.

4. The implant delivery system of claim 1, wherein the stop is configured to shift from a first configuration prior to passing through the aperture to a second configuration as it passes through the implant, wherein the first configuration is different from the second configuration.

5. The implant delivery system of claim 1, wherein the stop includes a node extending laterally away from the support member.

6. The implant delivery system of claim 1, wherein a distal tip portion of the support member extends beyond the node.

7. The implant delivery system of claim 6, wherein the distal tip portion of the support member is configured to be removed from the node subsequent to attaching the implant to the frame.

8. The implant delivery system of claim 7, wherein the node includes a first projecting portion extending a laterally away from a first lateral side of the support member and a second projecting portion extending laterally away from a second lateral side of the support member.

9. The implant delivery system of claim 1, wherein the stop includes an upper face configured to engage a bottom surface of the implant.

10. The implant delivery system of claim 1, wherein the stop includes a first support arm and a second support arm extending from the first support arm, and wherein the first support arm is positioned at an angle relative to the second support arm.

11. The implant delivery system of claim 1, wherein the stop is substantially U-shaped.

12. The implant delivery system of claim 1, wherein the implant is configured to be positioned between the stop and a distal portion of one of the plurality of attachment arms.

13. An implant delivery system, the implant delivery system comprising:
a tether including a proximal end and a distal end; and
a frame coupled to the distal end of the tether, the frame including a body portion and a plurality of attachment arms extending from the body portion, wherein each of the attachment arms includes a support member configured to extend through an aperture of an implant; and
wherein each of the attachment arms includes a stop disposed on a free end region of the support member, wherein the stop is configured to releasably secure the implant to the frame.

14. The implant delivery system of claim 13, wherein the stop is configured to engage a bottom surface of the implant to releasably secure the frame to the implant.

15. The implant delivery system of claim 13, wherein retraction of the tether is configured to pull the stop through the aperture of the implant.

16. The implant delivery system of claim 13, wherein the stop includes a node extending laterally away from the support member.

17. The implant delivery system of claim 16, wherein a distal tip portion of the support member extends beyond the node.

18. The implant delivery system of claim 17, wherein the distal tip portion of the support member is configured to be removed from the node subsequent to attaching the implant to the frame.

19. The implant delivery system of claim 13, wherein the stop includes a first support arm and a second support arm extending from the first support arm, and wherein the first support arm is positioned at an angle relative to the second support arm.

20. An implant delivery system, the implant delivery system comprising:
a frame including an upper surface and a bottom surface; and
a stabilizer coupled to the frame, wherein the stabilizer is positioned below the bottom surface of the frame, wherein the stabilizer is spaced away from the bottom surface of the frame to permit an implant to be releasably secured between the bottom surface of the frame and the stabilizer.
